(19) United States
(12) Patent Application Publication
(10) Pub. No.: US 2013/0064859 A1
(43) Pub. Date: Mar. 14, 2013

(54) COMBINATORIAL VITAMIN D AND PROBIOTIC THERAPY FOR INFLAMMATORY BOWEL DISEASE

(71) Applicants: Sarkis K. Mazmanian, Porter Ranch, CA (US); Martin Hewison, Calabasas, CA (US); Yue Shen, Albany, CA (US); Venu Lagishetty, Los Angeles, CA (US)

(72) Inventors: Sarkis K. Mazmanian, Porter Ranch, CA (US); Martin Hewison, Calabasas, CA (US); Yue Shen, Albany, CA (US); Venu Lagishetty, Los Angeles, CA (US)

(21) Appl. No.: 13/360,702

(22) Filed: Jan. 28, 2012

Related U.S. Application Data
(60) Provisional application No. 61/437,478, filed on Jan. 28, 2011.

(51) Int. Cl. A61K 35/74; A61P 37/04; A61P 3/02; C12N 5/0783; A61P 1/00; A61P 29/00

(52) U.S. Cl. 424/282.1; 435/355; 435/372.3

(57) ABSTRACT

A method of increasing the percentage of mouse and human Tregs is provided comprising providing B.fragilis or PSA and vitamin D or vitamin D metabolite to said mouse or human. Also, a method for treating inflammatory bowel diseases in an individual is presented where the individual is given a combination of vitamin D and B. fragilis or PSA. Such a treatment may be particularly effective in individuals who are either Vitamin D insufficient or deficient.

### RELATED APPLICATIONS

The present application claims the benefit of co-pending U.S. Provisional Application Ser. No. 61/437,478, filed on Jan. 28, 2011, which is incorporated herein by reference in its entirety for all purposes.

### BACKGROUND

1. Field of the Invention

The invention relates to methods of treating IBD; more specifically using a combination of probiotics and Vitamin D.

2. Related Art

Inflammatory bowel diseases (IBD) such as Crohn's disease and ulcerative colitis are prevalent and debilitating disorders of the intestinal tract. IBD affects an estimated 1 million people in the United States, and current therapies are often ineffective or have severe side-effects. Despite the social and medical burden of IBD, and significant basic and clinical research efforts, the incidence of Crohn's disease and ulcerative colitis continues to rise. The underlying cause of IBD remains enigmatic even after decades of research, but increasing evidence suggests that the pathophysiology of this disorder involves dysregulation of immune responses to intestinal bacteria. We have demonstrated that correction of aberrant mucosal inflammation is possible through the use of probiotic therapies derived from commensal gut bacteria.

Vitamin D is a potent modulator of innate and adaptive immunity. Epidemiology suggests that these two facets of vitamin D are linked, in that vitamin D-deficiency has been associated with a wide range of human diseases, notably IBD. There is widespread distribution of the vitamin D-activating enzyme CYP27B1 in normal human tissues [1]. The precursor of Vitamin D is 25-hydroxyvitamin D (25D), and the active form of Vitamin D is the 1,25-dihydroxyvitamin D (1,25D). Expression of the vitamin D-activating enzyme CYP27B1 has been detected in colonic epithelial cells [1], with CYP27B1 expression being altered in affected tissue from patients with Crohn's disease [2].

Clinically, probiotics alone and vitamin D supplementation alone have largely failed as viable IBD treatments.

### SUMMARY

PSA promotes colonic synthesis of active 1,25D from its 25D precursor, and 1,25D is a known activator of inducible Treg cells. The vitamin D pathway may be required, or may enhance, PSA-induced Treg development. The hypothesis to be tested here is that the beneficial effects of PSA require vitamin D activation, and that a combinatorial therapy will induce potent mouse and human Tregs and be effective in treating colitis in pre-clinical models of IBD.

In one embodiment, a method of increasing the percentage of mouse and human Tregs is provided comprising providing B.fragilis or PSA and vitamin D or vitamin D metabolite.

In another embodiment of the method of paragraph [0007], the vitamin D metabolite is 25D.

In a particular embodiment of paragraph [007], the method can be performed either in vitro or in vivo. When performed in vivo, the range of PSA can be 1 μg to 200 μg per mouse per treatment or 1 μg to 500 mg per human treatment; or 1×10⁴-1×10⁹ B. fragilis bacteria per treatment can be used; the range of Vitamin D (for 25-D); can be 1-1000 μg/Kg; and 0.1-50 μg/kg for 1,25 D. For in vitro, to naïve CD4+ T cells the range of PSA can be between 1 μg/mL to 200 μg/mL; vitamin D is 1-100 nM for 25-D and 0.1-10 nM for 1,25 D; the amount of B. fragilis can be determined or equivalent to the amount B. fragilis capble of producing the amount of PSA aforementioned.

In a particular example of the method of paragraph [007], mouse dendritic cells (DCs) are treated with PSA or PSA and vitamin D metabolite for 16-18 hrs, washed and co-cultured with naïve CD4+ T cells. After 4 days, cells were collected and stained for Foxp3. Percentage of Foxp3+ CD4+ T cells were determined by flow cytometry.

In another embodiment, a method of inducing Foxp3 expression in human Tregs is provided comprising providing B. fragilis or PSA. The method can be performed in vitro or in vivo. For in vitro, to naive CD4+ T cells the range of PSA can be between 1 μg/mL to 200 μg/mL; vitamin D is 1-100 nM for 25-D and 0.1-10 nM for 1,25 D; the amount of B. fragilis can be determined or equivalent to the amount B. fragilis capble of producing the amount of PSA aforementioned. For in vivo stimulation, range of PSA can be 1 μg to 200 μg per mouse treatment or 1 μg to 500 mg per human treatment; or 1×10⁴-1×10⁹ B. fragilis bacteria per treatment can be used; range of Vitamin D is 1-1000 μg/Kg for 25 D and 0.1-50 μg/Kg for 1,25 D. The required level of Foxp3 expression increased by such treatments is arbitrary and varies depending on context, and can be determined by a person of skill in the art. The level is increased compared to an untreated control group(s).

In a particular example of the method of paragraph [01], in vitro, human monocytes are treated with PSA or vehicle for 16-18 hrs, washed and co-cultured with CD4+ T cells from PBMCs of the same donor with or without TGFb. After 2 days, cells were collected and stained for Foxp3. MFI of Foxp3 among CD4+CD25+ T cells was determined by flow cytometry.

In another embodiment, a method of up-regulating expression of Cyp27b1 in colonic tissues is provided comprising providing PSA-expressing B. fragilis or PSA to an animal. The method can be performed in vitro or in vivo. For in vitro, the range of PSA can be between 1 μg/mL to 200 μg/mL; vitamin D is 1-100 nM for 25-D and 0.1-10 nM for 1,25 D; the amount of B. fragilis can be determined or equivalent to the amount B. fragilis capble of producing the amount of PSA aforementioned. For in vivo stimulation, range of PSA can be 1 μg to 200 μg per mouse treatment; 1 μg to 500 mg of PSA per human treatment; the amount of B. fragilis can be determined or equivalent to the amount B. fragilis capble of producing the amount of PSA aforementioned; or 1×10⁴-1×10⁹ B. fragilis bacteria per treatment can be used; range of Vitamin D is 1-1000 μg/Kg for 25 D and 0.1-50 μg/Kg for 1,25 D. The level of up regulation achieved is context dependent and can be determined by persons of skill in the art based on the increased level in synthesis of 1,25 D (or any Vitamin D type) required.

In an embodiment of the method of paragraph [0013] the up-regulating expression of Cyp27b1 results in promoting synthesis of 1,25D from the 25D precursor.

In an embodiment of the method of paragraph [0013] the up regulation occurs in dendritic cells (DCs).

In any of the aforementioned in vitro studies in paragraphs [007] thru [0013] where the combination of PSA or B. fragilis and Vitamin D and/or a Vitamin D metabolite is given, the combination can be added to naïve CD4 positive T cells.

In another embodiment, a method for treating inflammatory bowel diseases in an individual, comprising providing to said individual a combination of vitamin D and B. fragilis or PSA. The range of Vitamin D is 1-1000 μg/Kg for 25 D and 0.1-50 μg/Kg for 1,25 D; and 1 μg to 500 mg of PSA per human treatment; the amount of B. fragilis can be determined or equivalent to the amount B. fragilis capble of producing the amount of PSA aforementioned; or 1×10⁴-1×10⁹ B. fragilis bacteria per treatment can be used.

In an embodiment of the method of paragraph [0017], the individual is either vitamin D insufficient or deficient. In this respect, typically individuals who have 30 ng/mL or more 25 D are considered Vitamin D sufficient; individuals with 25 D levels of 21-29 ng/mL are considered Vitamin D insufficient; and individuals with 20 ng/mL of 25 D or less are considered deficient.

In yet another embodiment, a pharmaceutical composition for the treatment of IBD is provided comprising PSA or B. Fragilis and Vitamin D and/or a Vitamin D metabolite. In this respect, the range of Vitamin D can be 1-1000 μg/Kg for 25 D and 0.1-50 μg/Kg for 1,25 D; and 1 μg to 500 mg of PSA per human treatment; the amount of B. fragilis can be determined or equivalent to the amount B. fragilis capble of producing the amount of PSA aforementioned; or 1×10⁴-1×10⁹ B. fragilis bacteria per treatment can be used.

In an embodiment of the method of paragraph [0019], the pharmaceutical composition further comprises a conventional drug or treatment used for treating IBD and/or IBD symptoms.

### BRIEF DESCRIPTION OF THE DRAWINGS

For a more complete understanding of the present invention, reference is now made to the following descriptions taken in conjunction with the accompanying drawing, in which:

FIG. 1 is a schematic for interaction between PSA and the vitamin D system. FIG. 1 shows PSA, produced by B. fragilis, promotes synthesis of active 1,25D from the 25D precursor by activation of DCs. PSA signals through TLR2 on an intestinal DC to induce the expression of the vitamin D activating enzyme, Cyp27b1. This enzyme converts 25D to 1,25D, the active form of vitamin D, which binds to the vitamin D receptor (VDR) on naïve CD4+ T cells. In addition to other PSA-specific signals (reviewed in [3]), VDR-mediated gene expression promotes the development of regulatory T cells (Tregs). Both signals (PSA and 1,25D) are either required, or synergistically enhance, the induction of Tregs. PSA signals through TLR2 to induce CD4+Foxp3+ Tregs that express IL-10, leading to suppression of intestinal inflammation and colitis. Direct data for this model are provided in this application (see below). The proposed project will confirm this innovative process whereby PSA and vitamin D work in combination as an effective treatment for colitis by inducing the generation of Tregs cells that suppress intestinal inflammation.

FIG. 2 shows PSA expands functional Foxp3+ Tregs and cures experimental colitis. (A) Mice were treated with PSA or PBS. Analysis of the percentage of Foxp3+ cells within the CD4+CD25+ population in the MLNs is shown, indicated by the numbers in the top right quadrant. (B) CD4+CD25+ cells were purified from the MLNs of colitic PBS or PSA-treated mice and incubated with CFSE-pulsed CD4+CD25-responder cells in an in vitro suppression assay. Numbers indicate the percentage of cells undergoing at least one cellular division at 2 different ratios of effector T cells (Teff) and regulatory T cells (Treg). Top panels show results from healthy mice (−TNBS). (C) Disease scores for mice pre- and post-treated with PSA during TNBS colitis were assessed by a blinded pathologist. P values determined by a two-tailed Mann-Whitney U-test. **p value of <0.01. (D) Colons from mice were fixed, sectioned and stained with H&E to determine disease severity. Representative sections are shown.

FIG. 3 shows a protection from intestinal inflammation and colitis requires TLR2. (A) C57B1/6 WT or TLR2−/− animals were gavaged with purified PSA (or PBS) prior to TNBS administration. 5-6 days after onset of TNBS colitis, colons of animals were fixed, sectioned and H&E stained. Representative histological sections are shown. (B) Colitis scoring by a blinded pathologist based on a standard scoring system shows that PSA protects WT but not TLR2−/− animals from experimental colitis. Each symbol represents a separate animal. *p value of <0.05. NS, not significant. (C) RNA was extracted from the MLNs of indicated mice either treated with PBS or PSA, and the expression levels of IL-10 and IL-17A were assayed by qRT-PCR. (D) Percentages of CD4+CD25+Foxp3+ T cells in the MLNs of TLR2−/−mice treated with PBS or PSA. Plots are gated on CD4+ T cells. Note: PSA increases CD4+CD25+Foxp3+ Treg proportions in WT mice by ˜25%.

FIG. 4 shows Vitamin D-deficient mice are more susceptible to experimental colitis and harbor an increased bacterial burden, namely, Vitamin D-deficient mice have more gut bacteria, not the PSA producing bacteria in the colon. WT C57BL/6 mice raised on a vitamin D-deficient diet were induced for DSS-colitis (dextran sodium sulfate). (A) In the presence of DSS, the histological disease score is significantly increased when vitamin D levels are lower in animals, highlighting the protective effects of vitamin D. (B) qRT-PCR analysis of for the 16S small ribosomal subunit of bacteria from the colon reveals an increase in bacterial load during vitamin D restriction. These data reveal a link between increased colitis susceptibility and the microbiota during vitamin D deficiency.

FIG. 5 shows the effects of the microbiota on vitamin D metabolism in vivo. (A) qRT-PCR analysis of RNA from small intestine and kidney for Cyp27b1 in SPF (i.e. mice raised under normal specific pathogen free conditions) & GF (i.e. mice raised in sterile chambers) mice shows decreased expression of Cyp27b1 in the absence of microbiota. (B) GF animals also show reduced serum levels of 1,25D. **p<0.01, ***p<0.001. (C) Colonic Cyp27b1 mRNA in SPF, GF, and GF mice mono-colonized with B. fragilis (BF), or B. fragilis lacking PSA (BFΔPSA).

FIG. 6 shows Vitamin D augments the ability of PSA to promote mouse and human Tregs in vitro. (A) Mouse DCs were stimulated with PSA (or PBS control), and Cyp27b1 transcript levels were measured by qRT-PCR. PSA induces the expression of the vitamin D-activating enzyme from WT, but not TLR2−/− DCs. (B) Mouse DCs were treated with PSA, washed and co-cultured with naïve CD4+ T cells. After 4 days, PSA increased Foxp3+ Treg proportions (see % in box). The vitamin D metabolite 25D augmented PSA-induced Tregs. (C) Human monocytes were treated with PSA or vehicle, washed and co-cultured with CD4+ T cells from PBMCs of the same donor. The MFI for Foxp3 among CD4+CD25+ T cells is shown with or without TGFβ. Collectively, we propose a model whereby PSA signals through TLR2 on DCs to induce expression of Cyp27b1, which converts 25D to 1,25D. The vitamin D receptor (VDR) in T cells responds to 1,25 D, and along with other PSA-specific signals, mediated the development of Treg cells (see diagram in FIG. 1).

### DETAILED DESCRIPTION

The term “Vitamin D” as used herein includes any one or a combination +of a group of fat-soluble prohormones (D1-D5: 25 D, 1,25 D see below), which encourages the absorption and metabolism of calcium and phosphorous. Five forms of vitamin D have been discovered, vitamin D₁, D₂, D₃, D₄, D₅. The two forms that seem to matter to humans the most are vitamins D₂ (ergocalciferol) and D₃ (cholecalciferol), Vitamin D for humans is obtained from sun exposure, food and supplements. It is biologically inert and has to undergo two hydroxylation reactions to become active in the body. The term may also include 1,25-dihydroxycholecalciferol or 1,25-dihydroxyvitamin (“1,25-D”), which is considered the active form of vitamin D.

1,25 D is derived from its precursor 25-hydroxyvitamin-D(D-25) by the enzyme 1α-hydroxylase (“CYP27B1”) encoded by the CYP27B1 gene, (NG_{—}007076.1 Homo Sapiens) CYP27B1

The wording “polysaccharide A” (or PSA, or PSA ligand) as used herein indicates a molecule produced by the PSA locus of Bacteroides fragilis and derivatives thereof which include but are not limited to polymers of the repeating unit {→3) α-d-AAT Galp(1→4)-[β-d-Galf(1→3)] α-d-GalpNAc(1→3)-[4,6-pyruvate]-β-d-Galp(1→}, where AATGal is acetamido-amino-2,4,6-trideoxygalactose, and the galactopyranosyl residue is modified by a pyruvate substituent spanning O-4 and O-6. The term “derivative” as used herein with reference to a first polysaccharide (e.g., PSA), indicates a second polysaccharide that is structurally related to the first polysaccharide and is derivable from the first polysaccharide by a modification that introduces a feature that is not present in the first polysaccharide while retaining functional properties of the first polysaccharide. Accordingly, a derivative polysaccharide of PSA, usually differs from the original polysaccharide by modification of the repeating units or of the saccharidic component of one or more of the repeating units that might or might not be associated with an additional function not present in the original polysaccharide. A derivative polysaccharide of PSA retains however one or more functional activities that are herein described in connection with PSA in association with the anti-inflammatory activity of PSA.

The term “effective dose” is an amount that results in a reduction, inhibition or prevention of IBD and/symptoms associated with IBD in the individual. The amount of B. fragilis or other probiotic required to achieve this can be determined by a person of skill in the art.

The term “individual” as used herein includes a single biological organism wherein inflammation can occur including but not limited to animals and in particular higher animals and in particular vertebrates such as mammals and in particular human beings.

The term “individual in need of the treatment” means a person expressing or suffering from IBD and/symptoms related to IBD. An appropriately qualified person is able to identify such an individual in need of treatment using standard clinical protocols/guidelines. The same protocols/guidelines can also be used to determine whether there is improvement to the individual's disorder/symptoms; and determine the most effective dose of the B. fragilis, PSA, Vitamin D, or Vitamin D metabolite to give to an individual in need of the treatment.

The term “improvement” as used herein means a prevention or reduction in the severity or frequency, to whatever extent, of one or more symptoms associated with IBD in the individual. The improvement is either observed by the individual taking the treatment themselves or by another person (medical or otherwise).

The term “treatment” or “treating” as used herein indicates any activity that is part of a medical (prescribed by the physician) or non-medically approved (i.e. non-prescription including but not limited to vitamins, herbs; supplements; probiotics etc.) care that deals with IBD and/or symptom associated with IBD.

The term “prevention” as used herein indicates any activity that reduces the burden of the individual later expressing those behavioral symptoms. This takes place at primary, secondary and tertiary prevention levels, wherein: a) primary prevention avoids the development of IBD and/or its symptoms; b) secondary prevention activities are aimed at early stages of the IBD and/or its symptom treatment, thereby increasing opportunities for interventions to prevent progression of the IBD and/or its symptom and emergence of symptoms; and c) tertiary prevention reduces the negative impact of an already established condition of IBD and/or its symptom by restoring function and reducing any condition/disorder/symptom or related complications.

Pharmaceutically acceptable or appropriate carriers can be, but not limited to, organic or inorganic, solid or liquid excipient which is suitable for the selected mode of application such as oral application or injection, and administered in the form of a conventional pharmaceutical preparation. Such preparation includes solid such as tablets, granules, powders, capsules, and liquid such as solution, emulsion, suspension and the like. Said carrier includes starch, lactose, glucose, sucrose, dextrine, cellulose, paraffin, fatty acid glyceride, water, alcohol, gum arabic and the like. If necessary, auxiliary, stabilizer, emulsifier, lubricant, binder, pH adjustor controller, isotonic agent and other conventional additives may be added.

The pharmaceutically acceptable or appropriate carrier may well include other compounds known to be beneficial to an impaired situation of the GI tract, (e.g., antioxidants, such as Vitamin C, Vitamin E, Selenium or Zinc); or a food composition. The food composition can be, but is not limited to, milk, yoghurt, curd, cheese, fermented milks, milk based fermented products, ice-creams, fermented cereal based products, milk based powders, infant formulae, tablets, liquid bacterial suspensions, dried oral supplement, or wet oral supplement.

The term “nutraceutical” as used herein means a food stuff (as a fortified food or a dietary supplement) that provides health benefits. Nutraceutical foods are not subject to the same testing and regulations as pharmaceutical drugs.

The term “probiotic” as used herein means live microorganisms, which, when administered in adequate amounts, confer a health benefit on the host. The probiotics may be available in foods and dietary supplements (for example, but not limited to capsules, tablets, and powders). Examples of foods containing the probiotic are yogurt, fermented and unfermented milk, miso, tempeh, and some juices and soy beverages.

The term “extract” as used herein indicates either the insoluble material or soluble material obtained from the B. fragilis or related species using various chemical, immunological, biochemical or physical procedures known to those of skill in the art, including but not limited to, precipitation, centrifugation, filtering, column chromatography, and detergent lysis.

The term “whole cell lysate” refers to the fraction obtained when the B. fragilis or related species are lysed using detergent or other chemical or physical means.

The term “native” when used in connection with biological materials such as nucleic acid molecules, polypeptides, host cells, bacterial cells/strains and the like, refers to materials as they are found in nature and not manipulated by man.

The term “isolated” when used in connection biological materials such as nucleic acid molecules, polypeptides, bacterial cells, host cells, bacterial cells/strains and the like, refers to the isolated or purified aforementioned materials, where these materials do not occur naturally and/or where they have markedly different or distinctive characteristics compared to those found in the native material.

The term “non-denatured” is used herein refers to when the bacterial is frozen in a media and then undergoes a freeze-drying process. Such non-denatured bacteria can be mixed with other substances/compounds/carriers/additives and given in forms of a pill, tablet, or liquid to individuals in need of behavioral improvement. The non-denatured bacteria can also be mixed into foods (cookies, yogurt; milk etc.).

The term “conventional pharmaceuticals or compounds” as used herein in the context of other conventional pharmaceuticals or compounds used to treat IBD and/or its symptoms refers to those pharmaceuticals or compounds that persons of skill in the art (including but not limited to physicians) conventionally use to treat IBD and/or its symptoms.

It is further contemplated that the combination of PSA/B.fragilis and Vitamin D (or its metabolite) composition of the present invention can be used with one or more known medicaments known to be useful in the treatment or prevention of IBD and or its symptoms, either separately or in combination.

For example the compound/composition/pharmaceutical composition of the present invention can be combined with one or a combination of medicaments/treatments known to be useful in the treatment of IBD such as, but not limited to, sulfasalazine (Azulfadine), mesalamine (Asacol, Pentasa), immunosuppressants (Imuran, 6-MP, cyclosporine); methotrexate, TNF-alpha inhibitors (Remicade and Humira); and corticosteroids (Entocort and prednisone). Other treatments (experimental) for ulcerative colitis, include aloe vera, butyrate, boswellia, probiotics, antibiotics, immunosuppressive therapy, and nicotine.

More particularly, improvement in IBD encompasses a reduction in the severity or duration in any one or more clinical IBD symptoms, such as, but not limited to, Abdominal cramps and pain; Bloody stool; diarrhea; urgency to have a bowel movement; fever; loss of appetite; weight loss; mucus in the stool; ulceration of the large intestine; and anemia (due to blood loss). For example improvement in MS encompasses a reduction in the severity or duration in any one or more clinical MS symptoms, such as, but not limited to fatigue; visual disorders; numbness; dizziness/vertigo; bladder and bowel dysfunction; weakness; tremor; impaired mobility; sexual dysfunction; slurred speech; spasticity (leg stiffness); swallowing disorders; chronic aching pain; depression; mild cognitive and memory difficulties

Moreover, improved,” “improvement,” and other grammatical variants includes any change resulting in the reduction in the severity, duration, and/or risk of developing complications of the inflammatory disease. For IBD, ‘improved’ could mean for example, a reduced risk of the IBD subject developing profuse bleeding from the ulcers; perforation (rupture) of the bowel; strictures and obstruction; fistulae (abnormal passage) and perianal disease; toxic megacolon (acute nonobstructive dilation of the colon); and malignancy (for example, colon cancer).

The term “related” as used herein in the context of “B. fragilis and related species” refers to the other species under the genus Bacteroides (or otherwise), that were shown to have a positive effects on behaviors such as those tested here.

The term “induction” for instance, in the context of PSA-mediated induction of mouse and human Tregs, means an increase in the quantity and/or quality of mouse/human Tregs as determined by conventional quantitative or qualitative assays.

The term “enhancing” in the context of enhancing PSA-mediated induction of mouse and human Tregs means to any increase in the level of Tregs and/or increasing the intrinsic activity of Treg cells observed with PSA alone, compared to appropriate controls acceptable by those of skill in the art.

B. fragilis induces Tregs that prevent and cure experimental IBD. Maintenance of homeostasis toward the intestinal microbial milieu is critical to the preservation of health [3], and defects in mucosal tolerance lead to disorders such as IBD. In animal models of IBD, intestinal inflammation is suppressed by various subsets of regulatory T cells (Tregs) [4]. Tregs emerge from the thymus and are marked by expression of CD4, CD25 and the transcription factor Foxp3 (Forkhead Box transcription factor for Treg differentiation) [5]. We have recently reported that the prominent human commensal, B. fragilis, directs the development of Foxp3+ Tregs in the gut during protection from experimental colitis in animal models. Most significantly, the beneficial function of B. fragilis requires a single immunomodulatory molecule named Polysaccharide A (PSA). The Mazmanian laboratory employs 2 models of experimental colitis: 1) the T cell transfer model whereby various T cell subsets are transferred into RAG−/− animals allowing for the functional study of both pro- and anti-inflammatory T cells; 2) the TNBS (2,4,6-trinitrobenzene sulfonic acid) model, where rectal treatment of animals with an immunogenic chemical results in gut inflammation [6].

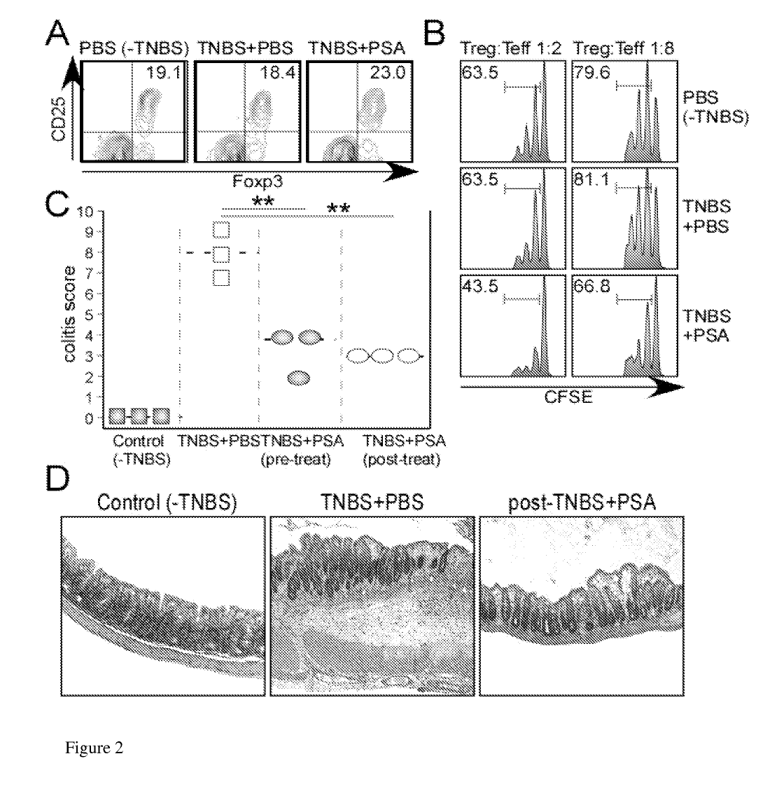

Here, we present data from the TNBS model, representative of results similar to the T cell transfer model. Vehicle treated (PBS; no TNBS) and PBS-treated TNBS animals (TNBS+PBS) had a similar percentage of Foxp3+ Treg cells within the CD4+CD25+ T cells of the gut (FIG. 2A). During protection from colitis (TNBS+PSA), PSA increases in-the percentage of Foxp3+ cells within the CD4+CD25+ compartment of the MLNs. Additionally, the absolute number of CD4+CD25+Foxp3+ cells in the MLNs was significantly higher in PSA-treated mice when compared to PBS or non-colitic animals (data not shown) [7]. The suppressive capacity of Tregs during PSA-mediated protection from intestinal inflammation was determined by in vitro suppression assays. As expected, proliferation of naïve T cells was partially suppressed when Tregs from vehicle (PBS) or PBS-treated colitic mice (TNBS+PBS) were added to the culture (FIG. 2B). Notably however, Tregs isolated from the MLNs of PSA fed mice (TNBS+PSA) suppressed T cell proliferation to a significantly higher level than cells from untreated animals, demonstrating that Tregs from animals protected from colitis by PSA have increased functional suppressive activity.

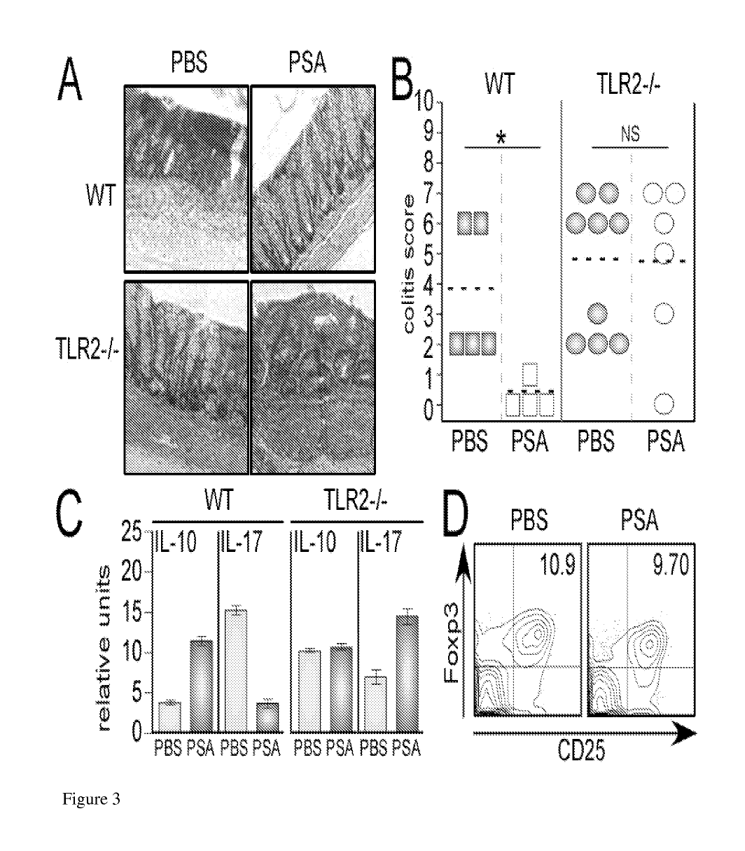

To examine if PSA can prevent and cure established colitis, animals were induced for TNBS colitis, and groups were either treated with vehicle (TNBS+PBS) or PSA either: 1) prior to disease induction (pre-TNBS+PSA) or 2) following rectal TNBS administration (post-TNBS+PSA). Using a standard scoring system, animals treated with TNBS showed a high degree of disease (FIG. 3C). However, both pre- and post-treatment with PSA prevented the development of disease with equal efficacy. Histologically, animals with colitis exhibited massive epithelial hyperplasia, thickening of the submucosa, and dramatic loss of colonocyte organization; features not seen in animals treated with PSA post-TNBS colitis (FIGS. 2C & D). Animals administered PSA following the onset of disease showed increased IL-10 and Foxp3 expression by gut CD4+ T cells, and a significant decrease in pro-inflammatory Th17 cells during both protection and cure of colitis (data not shown) [29].

PSA protection from experimental IBD requires TLR2 signaling. Multiple studies have implicated TLR signaling by innate immune cells during inflammatory disease [8]. As TLR2 has been shown to have significant effects on Treg expansion and function [9-10], we investigated if TLR2 is required for PSA activity. Wild-type (WT) or TLR2−/− animals were orally treated with PSA or PBS prior to induction of TNBS colitis. WT animals had clear histopathological signs of disease following administration of TNBS (PBS treatment), while WT animals treated with PSA were protected from disease (FIG. 3A). In contrast, TLR2−/− mice treated with PSA had marked inflammation including thickening of the mucosa, infiltrating leukocytes and crypt loss (FIG. 3A). TLR2−/− animals are not protected by PSA, as no differences in colitis scores were observed for TLR2 deficient animals treated with PBS or PSA (FIG. 3B). As previously reported [11], WT animals were protected from colitis by PSA. IL-10 expression was induced in response to PSA during protection from colitis in WT animals (FIG. 3C); however in the absence of TLR2 signaling, IL-10 was no longer up-regulated in PSA-treated animals. Suppression of the pro-inflammatory cytokine IL-17 by PSA was evident in WT animals; intriguingly, IL-17 levels were actually increased in TLR2−/− mice given PSA compared to PBS treatment, consistent with no protection from colitis in the absence of TLR2 (FIG. 3C). As shown in FIG. 3D, there are no differences in the percentage of CD4+Foxp3+ Tregs in TLR2−/− animals in response to PSA (relative to PBS controls). These results demonstrate that TLR2 signaling by PSA is required for Treg induction and for protection from experimental colitis.

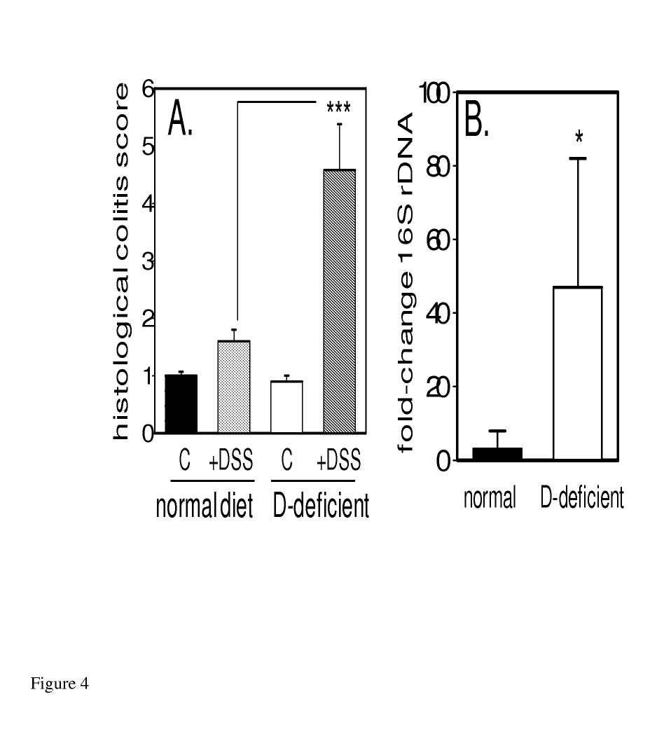

Increased IBD severity in vitamin D-deficient mice is associated with dysregulation of the microbiota. The protective role of vitamin D in pre-clinical models of IBD is now firmly established. Knockout of either the VDR [12, 13, 14] or Cyp27b1 [18] has been shown to increase the severity of colitis in mice. The Hewison group has shown that increased gastrointestinal inflammation and worse histological pathology is observed in vitamin D-deficient mice during experimental colitis (FIG. 4A) [15, 16]. These data support clinical and epidemiological findings from patients that vitamin D-deficiency is a feature of IBD. During colitis, bacteria are believed to promote intestinal inflammation and GF mice do not develop colitis in several animal models of IBD [3]. Intriguingly, analysis of the bacteria burden in the intestines of vitamin D-deficient animals shows a significant increase in bacterial load prior to the induction of experimental IBD (FIG. 4B). This finding serves to link the microbiota to the vitamin D system, and suggests that the interaction between the two may have important implications for IBD.

However, the effect of vitamin D-deficiency on the community composition of the microbiota remains unknown. Alterations in the profile of the microbiota, known as dysbiosis, are associated with IBD. To define a link between the composition of the microbiota and vitamin D, the current project proposes to extend these studies to comprehensively profile the microbiota of animals that are vitamin D-deficient due to dietary restriction, as well as due to genetic mutations in the vitamin D system.

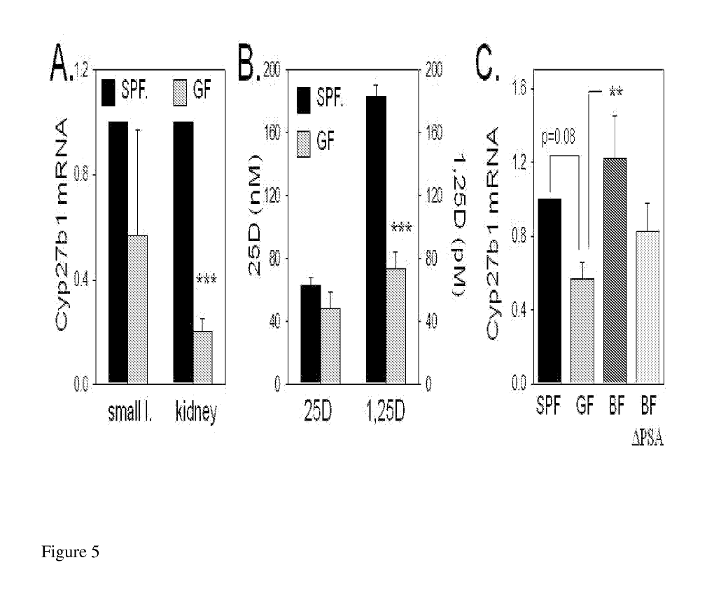

B. fragilis regulates renal and extra-renal metabolism of vitamin D. Previous studies by the Hewison group have documented expression of the vitamin D-activating enzyme CYP27B1 within its classical endocrine location in the kidney [17], as well as non-classical extra-renal sites such as the intestines [1]. In humans CYP27B1 is detectable in the colon [1], and we have shown that this expression is altered in patients with Crohn's disease [2]. More recently we have reported similar observations in colon tissue from mice with experimental IBD [18]. To specifically assess the impact of gut bacteria to the vitamin D system in the colon, we measured mRNA levels for Cyp27b1 and VDR in GF and specific pathogen free (SPF mice). Analysis of kidney tissue from GF mice showed decreased expression of Cyp27b1 and reduced serum levels of active 1,25D in the absence of the microbiota (FIG. 5A, 5B). By contrast, there was no significant change in vitamin D metabolism in the small intestine (a location with lower levels of microbiota compared to the colon) (FIG. 5A). The dramatically lowered serum concentrations of 1,25D (the active form of 25D) in GF mice represents a novel and remarkable phenotype linking gut bacteria to vitamin D status (FIG. 5B). In view of the strong association between the microbiota, vitamin D and IBD, we next wanted to determine if the vitamin D system is altered in the colon. We reveal data that Cyp27b1 expression is comparatively reduced in colonic tissues of GF mice (FIG. 5C; compare SPF and GF). Remarkably, mono-colonization of GF animals with B. fragilis restored Cyp27b1 expression in the colon to the high levels of SPF animals with a complex microbiota (FIG. 5C). This striking phenotype is dependent on PSA expression, as mono-colonization with B. fragilis lacking PSA (B. fragilisΔPSA) does not correct the decreased expression of Cyp27b1. Collectively, these data provide novel and important evidence that the microbiota profoundly influences the activation of vitamin D metabolites, and that B. fragilis activates the vitamin D system.

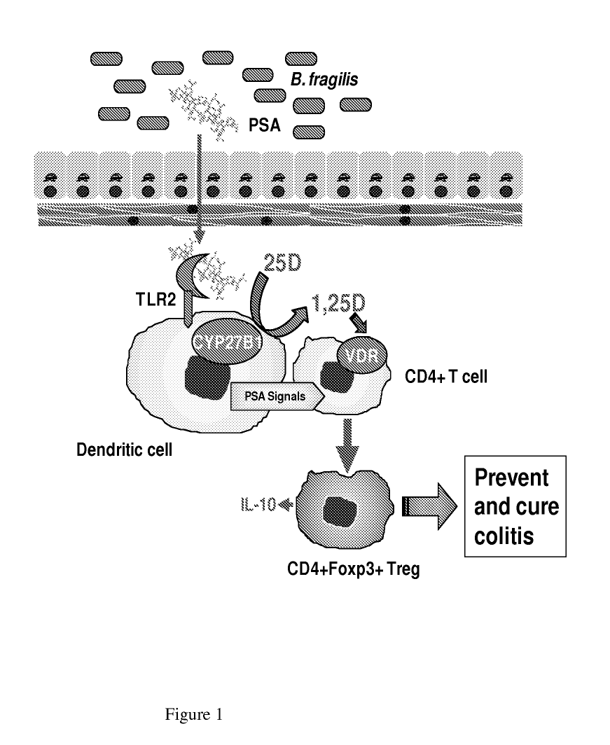

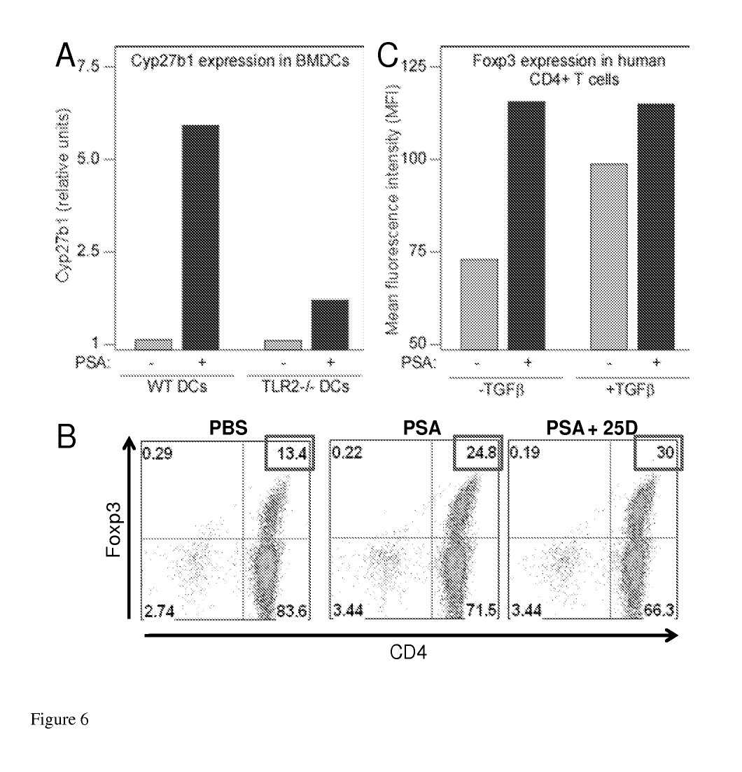

Vitamin D enhances PSA-mediated induction of mouse and human Tregs. As outlined above, the ability of PSA to protect against IBD in mice involves the gut-specific conversion of CD4+ cell into IL-10-secreting Foxp3+ Tregs [19]. We hypothesize that vitamin D may play a pivotal role in this process, given our novel finding that PSA induces the vitamin D-activating machinery in mice (see FIG. 5C). We therefore tested whether vitamin D enhanced the ability of PSA to induce the development of Foxp3+ Treg cells in vitro. Bone marrow-derived dendritic cells (BMDCs) from WT or TLR2−/− mice were treated with PSA (or PBS control). qRT-PCR analyses showed a 5-fold induction of Cyp27b1 expression in WT BMDC treated with PSA; importantly, this effect was greatly diminished in DCs from TLR2−/− mice (FIG. 6A). In subsequent studies, WT BMDCs treated with PSA with or without 25D were washed and incubated with CD4+ T cells harvested from the spleens of mice. FIG. 6B shows that PSA increases the proportions of CD4+Foxp3+ T cells in culture over media controls, with this effect being further enhanced by co-treatment with 25D. The Hewison group has previously reported TLR-inducible expression of CYP27B1 in human DCs [20], consistent with the data in shown FIG. 6A, and have also shown direct effects of vitamin D on human Treg development in vitro [21]. However, the ability of PSA to induce human Tregs has previously not been described. We now reveal that purified PSA promotes Foxp3 expression in human Tregs in vitro (FIG. 6C). TGFβ does not further enhance PSA-induced Tregs, demonstrating the potency of the PSA signal. Thus, for the first time, the anti-inflammatory effects of PSA are shown to translate to human cells. These studies firmly link PSA-induced Treg profiles to the vitamin D pathway, and demonstrate that this function may translate to human cells. As described in FIG. 1, we propose a model whereby PSA signals through TLR2 on dendritic cells to increase expression of the vitamin D activating enzyme, Cyp27b1, which results in conversion of 25D to the 1,25D active form. 1,25D then signals through the vitamin D receptor (VDR) in CD4+ T cells to induce Treg development (FIG. 2).

It should be understood that the above are merely examples and should not be used to limit how the methods can be utilized nor the claims.

### REFERENCES—WHICH ARE ALL INCLUDED BY REFERENCE IN THE ENTIRETY

1. Zehnder, D., et al., Extrarenal expression of 25-hydroxyvitamin d(3)-1 alpha-hydroxylase. J Clin Endocrinol Metab, 2001. 86(2): p. 888-94.
2. Abreu, M. T., et al., Measurement of vitamin D levels in inflammatory bowel disease patients reveals a subset of Crohn's disease patients with elevated 1,25-dihydroxyvitamin D and low bone mineral density. Gut, 2004. 53(8): p. 1129-36.
3. Round, J. L. and S. K. Mazmanian, The gut microbiota shapes intestinal immune responses during health and disease. Nat Rev Immunol, 2009.
4. Sakaguchi, S., et al., Foxp3+ CD25+ CD4+ natural regulatory T cells in dominant self-tolerance and autoimmune disease. Immunol Rev, 2006. 212: p. 8-27.
5. Fontenot, J. D., et al., Regulatory T cell lineage specification by the forkhead transcription factor foxp3. Immunity, 2005. 22(3): p. 329-41.
6. Neurath, M., I. Fuss, and W. Strober, TNBS-colitis. Int Rev Immunol, 2000. 19(1): p. 51-62.
7. Round, J. L. and S. K. Mazmanian, Inducible Foxp3+ regulatory T-cell development by a commensal bacterium of the intestinal microbiota.Proc Natl Acad Sci USA, 2010.
8. Strober, W., The multifaceted influence of the mucosal microflora on mucosal dendritic cell responses. Immunity, 2009. 31(3): p. 377-88.
9. Liu, H., et al., Toll-like receptor 2 signaling modulates the functions of CD4+ CD25+ regulatory T cells. Proc Natl Acad Sci USA, 2006. 103(18): p. 7048-53.
10. Sutmuller, R. P., et al., Toll-like receptor 2 controls expansion and function of regulatory T cells. J Clin Invest, 2006. 116(2): p. 485-94.
11. Mazmanian, S. K., J. L. Round, and D. L. Kasper, A microbial symbiosis factor prevents intestinal inflammatory disease. Nature, 2008. 453(7195): p. 620-5.
12. Froicu, M., et al., A crucial role for the vitamin D receptor in experimental inflammatory bowel diseases. Mol Endocrinol, 2003. 17(12): p. 2386-92.
13. Froicu, M., Y. Zhu, and M. T. Cantorna, Vitamin D receptor is required to control gastrointestinal immunity in IL-10 knockout mice. Immunology, 2006. 117(3): p. 310-8.
14. Kong, J., et al., Novel role of the vitamin D receptor in maintaining the integrity of the intestinal mucosal barrier. Am J Physiol Gastrointest Liver Physiol, 2008. 294(1): p. G208-16.
15. Lagishetty, V., et al., Vitamin D deficiency in mice impairs colonic antibacterial activity and predisposes to colitis. Endocrinology. 151(6): p. 2423-32.
16. Cantorna, M. T., et al., 1,25-Dihydroxycholecalciferol prevents and ameliorates symptoms of experimental murine inflammatory bowel disease. J Nutr, 2000. 130(11): p. 2648-52.
17. Zehnder, D., et al., Expression of 25-hydroxyvitamin D3-1 alpha-hydroxylase in the human kidney. J Am Soc Nephrol, 1999. 10(12): p. 2465-73.
18. Liu, N., et al., Altered endocrine and autocrine metabolism of vitamin D in a mouse model of gastrointestinal inflammation. Endocrinology, 2008. 149(10): p. 4799-808.
19. Round, J. L. and S. K. Mazmanian, Inducible Foxp3+ regulatory T-cell development by a commensal bacterium of the intestinal microbiota. Proc Natl Acad Sci USA. 107(27): p. 12204-9.
20. Hewison, M., et al., Differential regulation of vitamin D receptor and its ligand in human monocyte-derived dendritic cells. J Immunol, 2003. 170(11): p. 5382-90.
21. Jeffery, L. E., et al., 1,25-Dihydroxyvitamin D(3) and IL-2 combine to inhibit T cell production of inflammatory cytokines and promote development of regulatory T cells expressing CTLA-4 and FoxP3. J Immunol, 2009. 183(9): p. 5458-67.

What is claimed is:

1. A method of increasing the percentage of mouse and human Tregs comprising providing B.fragilis or PSA and vitamin D or vitamin D metabolite to said mouse or human.

2. The method of claim 1, wherein the method is performed in vitro or in vivo.

3. The method of claim 1, wherein the vitamin D metabolite is 25D.

4. The method of claim 2, wherein the method is performed in vitro on naïve CD4 positive T cells.

5. A method of inducing Foxp3 expression in human Tregs comprising providing B. fragilis or PSA.

6. The method of claim 5, wherein the promoting is performed either in vitro or in vivo.

7. The method of claim 5, wherein the method is performed in vitro on naïve CD4 positive T cells.

8. A method of up-regulating expression of Cyp27b1 in colonic tissues comprising providing PSA-expressing B. fragilis or PSA to an animal.

9. The method of claim 8, wherein the up-regulating expression of Cyp27b1 results in promoting synthesis of 1,25D from 25D precursor.

10. The method of claim 8, wherein the up regulation occurs in dendritic cells (DCs).

11. A method for treating inflammatory bowel diseases in an individual, comprising providing to said individual a combination of vitamin D and B. fragilis or PSA.

12. The method of claim 11, wherein the individual is vitamin D deficient.

13. The method of claim 11, wherein the individual is vitamin D insufficient.

14. A pharmaceutical composition for the treatment of IBD comprising PSA and Vitamin D and/or a Vitamin D metabolite.

15. The pharmaceutical composition of claim 14, further comprising a conventional drug or treatment used for treating IBD and/or IBD symptoms.
